(19) United States
(12) Patent Application Publication
(10) Pub. No.: US 2025/0243470 A1
(43) Pub. Date: Jul. 31, 2025

(54) Broad-Spectrum Phage for Efficiently Lysing Cronobacter, Bactericide, and Use

(71) Applicant: Hefei University of Technology, Hefei (CN)

(72) Inventors: Na LING, Hefei (CN); Bin CHENG, Hefei (CN); Yingwang YE, Hefei (CN); Ting WANG, Hefei (CN); Hanfang CHEN, Hefei (CN); Diwei ZHANG, Hefei (CN)

(21) Appl. No.: 18/561,084

(22) PCT Filed: Jun. 30, 2023
(86) PCT No.: PCT/CN2023/104443

§ 371 Date: Mar. 26, 2025

(51) Int. Cl. C12N 7/00; A01N 63/40; A01P 1/00; A23B 2/783

(52) U.S. Cl. CPC C12N 7/00; A01N 63/40; A01P 1/00; A23B 2/783; C12N 2795/00021; C12N 2795/00031; C12N 2795/00051

(57) ABSTRACT

Disclosed is a broad-spectrum phage for efficiently lysing Cronobacter, a bactericide, and use thereof. Also provided is a Cronobacter phage vB_CsaM_CBT2, where the phage has a deposit number of CCTCC NO: M 2023524. The phage only specifically lyses Cronobacter, has a broad lysis spectrum, and can cover four species of Cronobacter (including multi-drug-resistant bacteria). The phage shows a desirable stability at a pH value of 3 to 11 and 25° C. to 70° C., and has a bactericidal effect of 80.55% to 99.97% within 12 h in a milk powder sample. Moreover, the phage does not carry any virulence and antibiotic resistance genes, and meets the safety requirements in practical applications. Therefore, the phage provides a new strategy and resource guarantee for the control of Cronobacter contamination in powdered infant formula (PIF) and its industrial chain and environment, as well as the development of bactericides.

### TECHNICAL FIELD

The present disclosure belongs to the technical field of microorganisms, and in particular relates to a broad-spectrum phage for efficiently lysing Cronobacter, a bactericide, and use thereof.

### BACKGROUND

Cronobacter is an important foodborne pathogen. This genus can cause meningitis, sepsis, and necrotizing enterocolitis in neonates and infants, accompanied with neurologic sequelae in severe cases. Once the newborn is infected, there can be a mortality rate as high as 40% to 80%. Powdered infant formula (PIF) is considered to be a main route for the transmission of Cronobacter infection. The Cronobacter is highly resistant to environmental stress and can survive in the PIF for up to 2.5 years. Therefore, consumption of contaminated PIF carries a serious risk of fatal meningitis and intestinal infections. In 2004, the World Food and Agriculture Organization of the United Nations and the World Health Organization of the United Nations listed Cronobacter and Salmonella as Class A pathogenic bacteria of PIF through risk assessment. Since Cronobacter contamination incidents still occur from time to time, the efficient control of Cronobacter is a common problem that needs to be solved urgently in the dairy industry.

Antibiotics are currently the most effective treatment for diseases caused by Cronobacter infection. In recent years, research reports have confirmed that the drug resistance of Cronobacter spp. shows a trend of increasing year by year. Some research reports have pointed out that penicillin, first-generation and second-generation cephalosporins and other antibiotics commonly used in hospitals have lost their inhibitory effect on nosocomial infection of Cronobacter spp., and it is gradually found that there are Cronobacter isolates with multi-drug resistance spectrum. With the increasingly serious bacterial drug resistance, countries have strengthened the management of antibiotic abuse in recent years, and it is important to find antibacterial alternatives other than the antibiotics.

A phage is a virus that hosts a specific bacterial cell, injects its genetic materials into the host bacterium through a tail organelle, and then lyses the bacterium. The phages are harmless to humans, animals, and plants. The phage has abundant sources, high specificity, and high safety, and is an excellent substitute for antibiotics used in the control of food biological contamination. In order to effectively control Cronobacter contamination in food products, it is extremely urgent to screen a Cronobacter phage with a wide lysis spectrum.

### SUMMARY

An objective of the present disclosure is to provide a broad-spectrum phage for efficiently lysing Cronobacter, a bactericide, and use. The Cronobacter phage vB_CsaM_CBT2 can efficiently lyse multi-drug-resistant Cronobacter, and has a broad lysis spectrum.

The present disclosure provides a Cronobacter phage vB_CsaM_CBT2, where the phage has a deposit number of CCTCC NO: M 2023524.

The present disclosure further provides use of the Cronobacter phage vB_CsaM_CBT2 in production of a preparation for inhibiting and/or killing Cronobacter.

Preferably, the preparation is prepared in the form of a proliferation liquid of the Cronobacter phage vB_CsaM_CBT2 or a concentrate of the proliferation liquid.

Preferably, a preparation method of the proliferation liquid includes the following steps:
mixing a bacterial solution in a logarithmic phase of the Cronobacter and the Cronobacter phage vB_CsaM_CBT2 with an LB broth medium, conducting cell culture to obtain a culture solution; subjecting the culture solution to centrifugation to obtain a supernatant; and subjecting the supernatant to filtration to obtain the proliferation liquid of the Cronobacter phage vB_CsaM_CBT2.

Preferably, the Cronobacter is one or more selected from the group consisting of Cronobacter sakazakii, Cronobacter turicensis, Cronobacter muytjensii, and Cronobacter condimenti.

Preferably, the Cronobacter phage vB_CsaM_CBT2 in the preparation for inhibiting the Cronobacter has a working titer of greater than or equal to 2.95×10⁸ pfu/mL; and the Cronobacter phage vB_CsaM_CBT2 in the preparation for killing the Cronobacter has a working titer of greater than or equal to 1×10¹⁰ pfu/mL.

The present disclosure further provides an inhibitor or a bactericide of Cronobacter, including the Cronobacter phage vB_CsaM_CBT2 and a pharmaceutically acceptable excipient.

Preferably, the inhibitor or the bactericide of the Cronobacter has a working pH value of 3 to 11.

Preferably, the inhibitor of the Cronobacter has a working titer of greater than or equal to 2.95×10⁸ pfu/mL; and the bactericide of the Cronobacter has a working titer of greater than or equal to 1×10¹⁰ pfu/mL.

Preferably, the pharmaceutically acceptable excipient is one or more selected from the group consisting of a dispersant, a stabilizer, a filler, and a solvent.

The present disclosure further provides use of the Cronobacter phage vB_CsaM_CBT2 or the inhibitor or the bactericide of the Cronobacter in preparation of a reagent for controlling Cronobacter contamination.

Preferably, the reagent has a multiplicity of infection (MOI) of 1:10 to 1:1000.

Preferably, a process of controlling the Cronobacter contamination includes antagonizing the Cronobacter contamination in a product and/or an environment.

Preferably, the product includes a food product.

The present disclosure has beneficial effects as follows: the present disclosure provides a Cronobacter phage vB_CsaM_CBT2, where the phage has a deposit number of CCTCC NO: M 2023524. The Cronobacter phage vB_CsaM_CBT2 has a titer that can be maintained to not less than 10¹⁰ pfu/mL at 25° C. to 60° C. The Cronobacter phage vB_CsaM_CBT2 has a titer of not less than 10⁸ pfu/mL at a pH value of 4 to 11. The Cronobacter phage vB_CsaM_CBT2 has a high titer, high temperature and pH stability, and a desirable antibacterial effect.

In the present disclosure, the Cronobacter phage vB_CsaM_CBT2 has a broad host spectrum and can target and lyse four strains of C. sakazakii, C. turicensis, C. muytjensii, and C. condimenti in the genus Cronobacter, showing a broad lysis spectrum. The Cronobacter phage vB_CsaM_CBT2 can lyse a multi-drug-resistant clinical strain GZcsf-1 of the C. sakazakii. The clinical strain GZcsf-1 of the C. sakazakii can tolerate antibiotics such as ampicillin, cefazolin, ceftriaxone, aztreonam, gentamicin, tetracycline, chloramphenicol, and methoxypyridazine/sulfamethoxazole.

### Deposit of Biological Material

The Cronobacter phage vB_CsaM_CBT2 was deposited at the China Center for Type Culture Collection (CCTCC), No. 299, Bayi Road, Wuchang District, Wuhan City, Hubei Province on Apr. 12, 2023 with a deposit number of CCTCC NO: M 2023524.

### BRIEF DESCRIPTION OF THE DRAWINGS

To illustrate the examples of the present disclosure or the technical solutions in the prior art more clearly, the accompanying drawings required in the examples will be briefly introduced below.

FIG. 1 shows a phage plaque picture of the Cronobacter phage vB_CsaM_CBT2;

FIG. 2 shows an electron micrograph of the Cronobacter phage vB_CsaM_CBT2;

FIG. 3 shows a genome analysis heat map of the Cronobacter phage vB_CsaM_CBT2;

FIG. 4 shows an analysis diagram of a phylogenetic tree of a terminal enzyme large subunit of the Cronobacter phage vB_CsaM_CBT2;

FIG. 5 shows a schematic diagram of a one-step growth curve of the Cronobacter phage vB_CsaM_CBT2;

FIG. 6 shows a schematic diagram of temperature tolerance of the Cronobacter phage vB_CsaM_CBT2; and

FIG. 7 shows a schematic diagram of pH tolerance of the Cronobacter phage vB_CsaM_CBT2.

### DETAILED DESCRIPTION OF THE EMBODIMENTS

The present disclosure provides a Cronobacter phage vB_CsaM_CBT2, where the phage has a deposit number of CCTCC NO: M 2023524.

In the present disclosure, a potent Cronobacter phage vB_CsaM_CBT2 (hereinafter referred to as Cronobacter phage vB_CsaM_CBT2) that efficiently lyses multi-drug-resistant bacteria is selected and isolated from the sewage of a sewage treatment plant in Hefei City, Anhui Province. Whole-genome analysis is conducted on the Cronobacter phage vB_CsaM_CBT2, and the Cronobacter phage vB_CsaM_CBT2 is a member of Straboviridae and is classified as Cronobacter phage. A genome of the Cronobacter phage vB_CsaM_CBT2 has a full length of 179,308 bp and 278 coding sequences. The Cronobacter phage vB_CsaM_CBT2 has polyhedral head and tail structures; the head has a length of about 105 nm and a width of about 72 nm, and the tail has a length of about 110 nm.

In the present disclosure, through the research on a performance of the Cronobacter phage vB_CsaM_CBT2, it is found that the phage has a wide host spectrum, well temperature and pH stability, and desirable antibacterial effect. In the present disclosure, the Cronobacter phage vB_CsaM_CBT2 has a broad host spectrum and can target and lyse 17 strains from four species of C. sakazakii, C. turicensis, C. muytjensii, and C. condimenti in the genus Cronobacter. The phage can provide resources for the control of clinical strains of multi-drug-resistant Cronobacter. The Cronobacter phage vB_CsaM_CBT2 does not contain bacterial drug resistance and virulence genes, has high safety, and can be applied to the control of Cronobacter in food.

In the present disclosure, the Cronobacter phage vB_CsaM_CBT2 has a working pH value of preferably 3 to 11, more preferably 5 to 10, more preferably 6 to 9. The Cronobacter phage has a working temperature of preferably 25° C. to 60° C., more preferably 25° C. to 50° C., even more preferably 25° C. to 40° C., and the most preferably 25° C.

In the present disclosure, the pH value and temperature are set to improve a lysis effect of the Cronobacter phage on Cronobacter.

The present disclosure further provides use of the Cronobacter phage in production of a preparation for inhibiting and/or killing Cronobacter.

In the present disclosure, the preparation for inhibiting and/or killing the Cronobacter preferably includes a preparation for inhibiting or killing the Cronobacter in the form of a proliferation liquid of the Cronobacter phage vB_CsaM_CBT2 or a concentrate of the proliferation liquid of the Cronobacter phage vB_CsaM_CBT2. A preparation method of the proliferation liquid of the Cronobacter phage vB_CsaM_CBT2 includes preferably the following steps:
subjecting Cronobacter to first culture in an LB broth medium, and collecting a bacterial solution in a logarithmic phase of the Cronobacter; and
mixing the bacterial solution in the logarithmic phase of the Cronobacter and the Cronobacter phage vB_CsaM_CBT2 with the LB broth medium, conducting cell culture to obtain a culture solution; subjecting the culture solution to centrifugation to obtain a supernatant; and subjecting the supernatant to filtration to obtain the proliferation liquid of the Cronobacter phage vB_CsaM_CBT2.

In the present disclosure, Cronobacter is subjected to first culture in an LB broth medium, and a bacterial solution in a logarithmic phase of the Cronobacter is collected. The first culture is conducted at preferably 35° C. to 40° C., more preferably at 33° C. to 39° C., and even more preferably at 37° C. The Cronobacter is preferably Cronobacter GZcsf-1.

In the present disclosure, after the first culture is completed, the bacterial solution in the logarithmic phase of the Cronobacter is collected; preferably, the culture solution is obtained by mixing the bacterial solution in the logarithmic phase of the Cronobacter and the Cronobacter phage vB_CsaM_CBT2 with the LB broth medium. The second culture is conducted at preferably 30° C. to 40° C., more preferably 32° C. to 38° C., and even more preferably at 37° C. The second culture is conducted for preferably 2 h to 6 h, more preferably 4 h. The bacterial solution in the logarithmic phase of the Cronobacter, the Cronobacter phage, and the LB broth medium are at a volume ratio of preferably (1-2):(1-2): 50, more preferably 1:1:50. The Cronobacter phage and the bacterial solution of the Cronobacter are at a viable count ratio of preferably 1:10 to 1:1000, more preferably 1:70 to 1:500, even more preferably 1:80 to 1:200, and most preferably 1:100.

In the present disclosure, after the culture solution is obtained, the culture solution is preferably centrifuged to obtain the supernatant; the supernatant is filtered to obtain the proliferation liquid of the Cronobacter phage. The centrifugation is conducted at preferably 3,800 g to 4,200 g, more preferably 4,000 g for preferably 15 min to 20 min, more preferably 18 min. The filtration is preferably effected by a filter membrane having a pore size of preferably 0.45 μm. The Cronobacter phage in the proliferation liquid of the Cronobacter phage has a working titer of preferably greater than or equal to 2.95×10⁸ pfu/mL, more preferably 6.40×10⁹ pfu/mL to 1.18×10¹⁰ pfu/mL, even more preferably 1.13×10¹⁰ pfu/mL to 1.18×10¹⁰ pfu/mL, and most preferably 1.18×10¹⁰ pfu/mL.

In the present disclosure, after the proliferation liquid of Cronobacter phage is obtained, NaCl and polyethylene glycol 8000 are preferably added to the proliferation liquid of the Cronobacter phage, and a precipitate is obtained by centrifugation; the precipitate is mixed with an SM buffer to obtain the concentrate of the Cronobacter phage.

In the present disclosure, the LB broth medium preferably uses distilled water as a solvent, and includes the following components by concentration: 10 g/L of tryptone, 5 g/L of a yeast extract, and 10 g/L of sodium chloride, and has a pH value of 6.9 to 7.1.

In the present disclosure, the double LB broth medium preferably uses distilled water as a solvent, and includes the following components by concentration: 20 g/L of tryptone, 10 g/L of a yeast extract, and 20 g/L of sodium chloride, and has a pH value of 6.9 to 7.1.

In the present disclosure, after the proliferation liquid of the Cronobacter phage or the concentrate of the phage is obtained, a preparation for inhibiting or killing the Cronobacter is prepared. The Cronobacter phage vB_CsaM_CBT2 in the preparation for inhibiting the Cronobacter has a working titer of greater than or equal to 2.95×10⁸ pfu/mL, more preferably greater than or equal to 1×10⁹ pfu/mL.

In the present disclosure, the Cronobacter phage vB_CsaM_CBT2 in the preparation for killing the Cronobacter has a working titer of greater than or equal to 1×10¹⁰ pfu/mL, more preferably greater than or equal to 1.18×10¹⁰ pfu/mL.

In the present disclosure, the Cronobacter inhibited and/or killed by the Cronobacter phage is preferably one or more selected from the group consisting of C. sakazakii, C. turicensis, C. muytjensii, and C. condimenti, more preferably the C. sakazakii, the C. turicensis, the C. muytjensii, and the C. condimenti.

The present disclosure further provides an inhibitor or a bactericide of Cronobacter, including the Cronobacter phage vB_CsaM_CBT2 and a pharmaceutically acceptable excipient. In the present disclosure, the inhibitor or the bactericide of the Cronobacter has a working pH value of preferably 3 to 11, more preferably 5 to 10, more preferably 6 to 9. The inhibitor of the Cronobacter phage has a working titer of preferably greater than or equal to 2.95×10⁸ pfu/mL, more preferably 1×10⁹ pfu/mL; and the bactericide of the Cronobacter has a working titer of preferably greater than or equal to 1×10¹⁰ pfu/mL, more preferably 1.18×10¹⁰ pfu/mL.

In the present disclosure, the pharmaceutically acceptable excipient includes preferably one or more of a dispersant, a stabilizer, a filler, and a solvent.

The present disclosure further provides use of the Cronobacter phage vB_CsaM_CBT2 or the inhibitor or the bactericide of the Cronobacter in preparation of a reagent for controlling Cronobacter contamination.

In the present disclosure, the use includes preferably the following steps: mixing the Cronobacter phage vB_CsaM_CBT2 or the inhibitor or the bactericide of the Cronobacter with a sample containing the Cronobacter to allow inhibiting or killing; more preferably, mixing the proliferation liquid or the concentrate of the Cronobacter phage with the sample containing the Cronobacter to allow inhibiting or killing.

In the present disclosure, a preparation method of the proliferation liquid or the concentrate of the Cronobacter phage has been discussed above and will not be repeated here.

In the present disclosure, there is no special limitation on a mixing method, and conventional methods can be used.

In the present disclosure, a process of controlling the Cronobacter contamination includes antagonizing the Cronobacter contamination in a product and/or an environment. The product is preferably a food product; and the food product includes preferably infant food, more preferably powdered infant formula (PIF).

In the present disclosure, the reagent has an MOI of preferably 1:10 to 1:1000, more preferably 1:70 to 1:500, even more preferably 1:80 to 1:200, and most preferably 1:100.

In the present disclosure, the MOI of the Cronobacter phage is set to improve a lysis effect of the Cronobacter phage on the Cronobacter.

In the present disclosure, the Cronobacter phage vB_CsaM_CBT2 has no obvious change in titer under different conditions of temperature at 25° C. to 70° C. and pH value at 3 to 11. The Cronobacter phage vB_CsaM_CBT2 has a titer that can be maintained to not less than 10¹⁰ pfu/mL at 25° C. to 60° C. The Cronobacter phage vB_CsaM_CBT2 has a titer of not less than 10⁸ pfu/mL at a pH value of 4 to 11. The Cronobacter phage vB_CsaM_CBT2 has a titer of 4.30×10⁷ pfu/mL after incubation at a pH value of 3 for 1 h, and a titer of 3.27×10⁸ pfu/mL after incubation at a pH value of 11 for 1 h; the Cronobacter phage vB_CsaM_CBT2 has a titer of 2.827×10⁸ pfu/mL after incubation at 65° C. for 1 h. The Cronobacter phage vB_CsaM_CBT2 has a titer of 2.60×10⁸ pfu/mL after incubation at 70° C. for 1 h.

In the present disclosure, the Cronobacter phage vB_CsaM_CBT2 only specifically lyses the Cronobacter, and can target and lyse strains from four species of multi-drug-resistant Cronobacter, including C. sakazakii, C. turicensis, C. muytjensii, and C. condimenti in the genus Cronobacter. The Cronobacter phage vB_CsaM_CBT2 has desirable stability under the conditions of pH value at 3 to 11 and temperature at 25° C. to 70° C., and shows a well bactericidal effect. Moreover, the phage does not carry any virulence and antibiotic resistance genes, and meets the safety requirements in practical applications. Therefore, the phage provides innovation strategies and valuable resources for the control of Cronobacter contamination in PIF and its industrial chain and environment.

The Cronobacter phage vB_CsaM_CBT2 can kill Cronobacter GZcsf-1 in a milk powder, showing that a bactericidal effect in milk powder samples is as high as 80.55% to 99.97% within 12 h. Therefore, the phage provides a new strategy and resource guarantee for the control of Cronobacter contamination in PIF and its industrial chain and environment, as well as the development of bactericides.

In order to further illustrate the present disclosure, the technical solutions provided by the present disclosure will be described in detail below in conjunction with accompanying drawings and examples, but they should not be construed as limiting the protection scope of the present disclosure.

Media used in the following examples were:

LB broth medium with distilled water as a solvent, containing: 10 g/L of tryptone, 5 g/L of yeast extract, and 10 g/L of sodium chloride, and having a pH value of 6.9 to 7.1.

Double LB broth medium with distilled water as a solvent, containing: 20 g/L of tryptone, 10 g/L of yeast extract, and 20 g/L of sodium chloride, and having a pH value of 6.9 to 7.1.

CaCl₂) was added to the double LB broth medium to a final concentration of 2 mM during use.

0.7% LB-Ca soft agar medium: CaCl₂) with a final concentration of 2 mM added into LB soft agar with an agar mass concentration of 0.7%.

1.5% LB agar medium: LB agar medium with an agar mass concentration of 1.5%.

0.4% LB-Ca soft agar medium: CaCl₂) with a final concentration of 2 mM added into LB soft agar with an agar mass concentration of 0.4%.

### Example 1 Isolation and Purification of Phage

### 1. Treatment of Water Sample

(1) Water sample was the sewage collected from a sewage treatment plant in Hefei City, Anhui Province in 2022.
(2) The water sample was centrifuged at 10,000 g for 10 min; large solid particles and some bacteria were removed by the centrifugation to obtain a supernatant; the supernatant was vacuum-filtered with a 0.45 μm aqueous filter membrane into a clean and sterile Erlenmeyer flask. MgSO₄ was added to the substances in Erlenmeyer flask to a final concentration of 50 mM, stirred evenly, and allowed to stand for 15 min to 20 min to obtain a first mixed solution. The first mixed solution was vacuum filtered, a supernatant was discarded, and a filter membrane was collected. The filter membrane was cut into pieces and put into a beaker with an appropriate amount of eluent, and ultrasonicated for 4 min to 5 min to elute phage particles from the filter membrane to obtain a second mixed solution. The second mixed solution was suction-filtered into another dry and sterile Erlenmeyer flask with a filter head of 0.45 μm, and this liquid was a phage mixed solution. The phage mixed solution was stored at 4° C. for future use.
(3) Cronobacter GZcsf-1 was inoculated into 5 mL of an LB broth medium under sterile conditions, and cultured overnight to obtain an activated bacterial solution. The bacterial solution was inoculated into a fresh 5 mL LB broth medium with an inoculum volume of 1% by volume and cultured to a logarithmic phase to obtain a bacterial solution in the logarithmic phase of the Cronobacter GZcsf-1.
(4) 500 μL of the phage mixed solution obtained in step (2), 500 μL of double LB broth medium (containing CaCl₂) at a final concentration of 2 mM), and 50 μL of bacterial solution in the logarithmic phase of the Cronobacter GZcsf-1 obtained in step (3) were mixed evenly, and cultured with shaking at 37° C. for 8 h to obtain a culture solution. The culture solution was centrifuged at 4,000 g for 15 min to 20 min to obtain a supernatant, which was suction-filtered with a 0.45 μm filter head into a sterile centrifuge tube to obtain a phage lysate. The phage lysate was stored at 4° C. for future use.

### 2. Purification of Phage

A 1.5% LB agar medium was poured on a clean and sterile petri dish. 100 μL of the bacterial solution in the logarithmic phase of the Cronobacter GZesf-1 and 100 μL of the phage lysate were added into 5 mL of a 0.7% LB-Ca soft agar medium, and mixed well. A resulting mixture was spread on the surface of a dry 1.5% LB agar medium, and then incubated at 37° C. for 8 h to obtain clearly visible phage plaques.

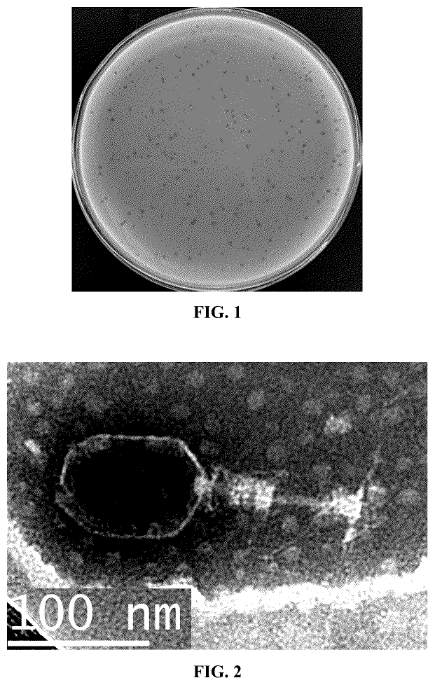

A single transparent phage plaque was picked with a small sterile pipette tip and dissolved in an appropriate amount of SM buffer, mixed well, centrifuged at 4,000 g for 15 min to 20 min, and a supernatant was collected. The supernatant was suction-filtered with a 0.45 μm filter head to obtain a purified phage solution. The purified phage solution was purified repeatedly until the phage plaque was single and transparent (FIG. 1), and a Cronobacter phage was isolated and named vB_CsaM_CBT2.

### 3. Proliferation and Concentration of Phage

1 mL of LB-Ca broth medium was added into a sterile centrifuge tube, 20 μL of the bacterial solution of the Cronobacter GZcsf-1 cultured to the logarithmic phase and 20 μL of the purified phage solution were added, and incubated at 37° C., 200 rpm for 4 h. An obtained culture solution was centrifuged at 4,000 g for 15 min to 20 min, and a supernatant was suction-filtered with a 0.45 μm filter head into a new sterile centrifuge tube to obtain a phage proliferation liquid.

NaCl and polyethylene glycol 8000 were added into the phage proliferation liquid, where NaCl was added to a final concentration of 0.5M and the polyethylene glycol 8000 was added to a final concentration of 10%. A resulting mixture was allowed to settle the phage particles overnight at 4° C., and then centrifuged at 12,000 g for 10 min to remove a supernatant, and a precipitate was collected. The precipitate was redissolved in SM buffer to obtain a phage concentrate. The phage concentrate was stored at 4° C. for future use. (For long-term storage, the phage concentrate was added with glycerol at a final concentration of 30% and stored at −20° C.).

### Example 2 Morphological Observation of Phage

The purified solution of phage vB_CsaM_CBT2 obtained in Example 1 was dripped on a copper grid, absorbed with filter paper after 3 min to 5 min, stained with a drop of phosphotungstic acid (PTA) for 2 min to 3 min, the excess liquid was removed by absorption, and dried in the air. The phage vB_CsaM_CBT2 was observed by a field emission transmission electron microscope, the head had a length of about 105 nm and a width of about 72 nm, and the tail had a length of about 110 nm (FIG. 2).

### Example 3 Genome Sequencing and Analysis of Phage

### 1. Extraction of Phage DNA

(1) DNase I and RNase A to 500 μL of the phage concentrate obtained in Example 1, incubated at 37° C. for 60 min (inverted once every 30 min), then added with 10% SDS, proteinase K, and EDTA, incubated at 65° C. for 60 min (inverted once every 30 min), then added with an equal volume of Tris-saturated phenol, vortexed for 30 s, and centrifuged at 12,000 g for 10 min to obtain an upper aqueous phase.
(2) the upper aqueous phase was absorbed into a new centrifuge tube; a phenol-chloroform-isoamyl alcohol mixture with an equal volume to water was added, vortexed for 30 s, and centrifuged at 12,000 g for 10 min to obtain an upper aqueous phase; where phenol, chloroform, and isoamyl alcohol in the mixture were at a volume ratio of 25:24:1.
(3) The upper aqueous phase was pipetted into a new centrifuge tube, then added with an equal volume of chloroform and vortexed for 30 s to mix the liquid evenly, then centrifuged at 12,000 g for 10 min to obtain an upper aqueous phase. The chloroform extraction was repeated twice to obtain an upper aqueous phase.
(4) An equal volume of isopropanol was added to the upper aqueous phase, mixed well, placed at −20° C. for 30 min, and centrifuged at 12,000 g for 20 min at 4° C. to collect a precipitate.
(5) The precipitate was washed with 200 μL of 70% ethanol, centrifuged at 12,000 g for 5 min, a supernatant was discarded, and a DNA precipitate was retained. The ethanol washing was repeated twice.
(6) The DNA precipitate was dried at room temperature, added with 20 μL of sterile water preheated at 65° C., and stored at −20° C. for later use.

### 2. Whole-Genome Analysis

Phage DNA was subjected to whole-genome sequencing using Illumina NextSeq sequencer. According to the Illumina sequencing results, the phage vB_CsaM_CBT2 was a linear, double-stranded DNA (dsDNA) phage. The phage vB_CsaM_CBT2 had a complete genome sequence length of 179,308 bp, a GC content of 44.83%, and contains 278 coding sequences. The similarities between the phage vB_CsaM_CBT2 and Enterobacter phage vB_EkoM5VN and Cronobacter phage vB_CsaM_1eN were 95.4% and 95.3%, respectively.

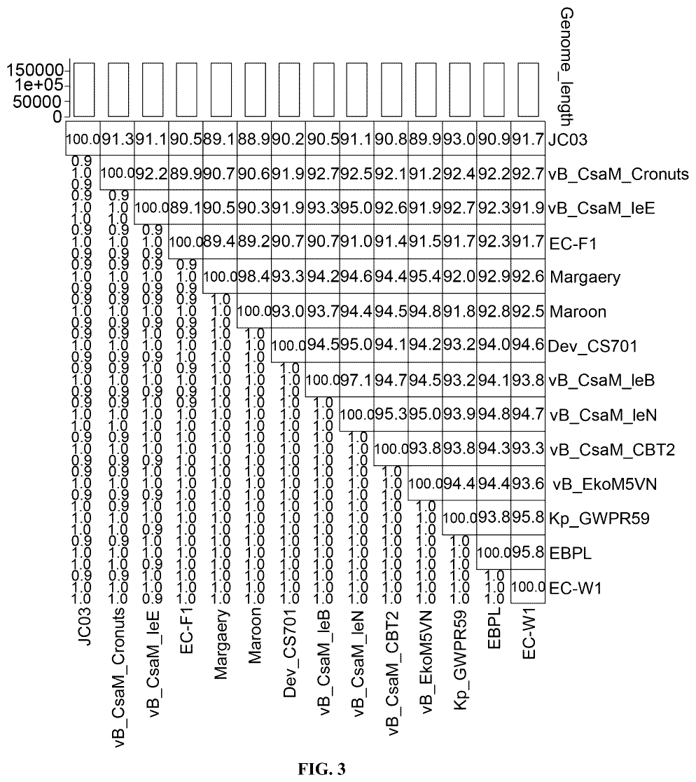

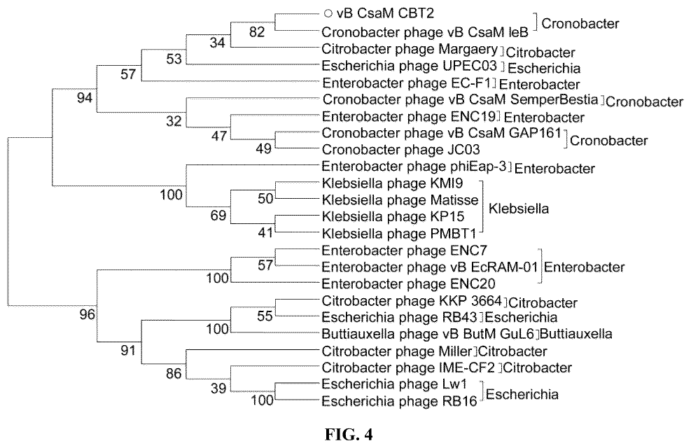

Through NCBI database comparison, NCBI showed that phages vB_EkoM5VN and vB_CsaM_1eN belonged to Straboviridae, vB_CsaM_CBT2 belonged to a member of Straboviridae, and was classified as Cronobacter phage, that is, the Cronobacter phage vB_CsaM_CBT2. The genome analysis heat map (FIG. 3) and the analysis map of the phylogenetic tree of the terminal enzyme large subunit (FIG. 4) of the Cronobacter phage vB_CsaM_CBT2 also supported this conclusion, showing that the vB_CsaM_CBT2 was a Cronobacter phage.

Compared with the virulence database and antibiotic resistance gene database, the phage vB_CsaM_CBT2 did not contain virulence and antibiotic genes, indicating that it could be safely used to control Cronobacter.

### Example 4 Determination of a Host Spectrum of Cronobacter Phage vB_CsaM_CBT2

A 1.5% LB agar medium was spread onto a dry sterile petri dish and allowed to dry. A bacterial solution in a logarithmic phase of the bacteria numbered 1 to 60 in Table 1 was separately prepared. 100 μL of each bacterial solution cultured to the logarithmic phase was added into 5 mL of 0.4% LB-Ca soft agar medium, mixed well, spread on a dried plate, and dried naturally to solidify the soft agar medium. 2 μL of the Cronobacter phage vB_CsaM_CBT2 concentrate prepared in Example 1 was added to the soft agar by spotting, dried naturally, and incubated at 37° C. for 4 h to 6 h. The culture results were divided into two classes: clear spotting area (+) and plaque free in spotting area (−).

The results are shown in Table 1. 19 strains of six species of Cronobacter and 41 strains of non-Cronobacter were selected. The Cronobacter phage vB_CsaM_CBT2 only specifically lysed Cronobacter, and lysed a total of 17 strains from four species of Cronobacter, including C. sakazakii, C. turicensis, C. muytjensii, and C. condimenti. The serial numbers 1 to 13 belonged to the C. sakazakii species, the serial numbers 14 to 15 belong to the C. turicensis species, the serial numbers 16 belonged to the C. muytjensii species, and the serial numbers 17 belonged to the C. condimenti species. The phage showed no lytic effect on the non-Cronobacter strains.

In the present disclosure, there was no limitation on the source of the strain for verifying the effect of Cronobacter phage vB_CsaM_CBT2, and conventional strains could be used.

In this example, there were 60 bacterial strains in Table 1. The selected 19 strains of Cronobacter were preserved by the laboratory of Hefei University of Technology, among which LMG 26250 and NCTC 9529 were the standard strains. The selected 10 strains were Burkholderia gladioli, among which ATCC 33664 was the standard strain, and the remaining 9 strains were Burkholderia gladioli isolated and identified from black fungus, Flammulina velutipes, and shiitake mushrooms purchased in various vegetable markets in Hefei from 2021 to 2022. The selected 10 strains of Vibrio parahaemolyticus were isolated from food by Guangdong Institute of Microbiology. The remaining 21 strains were preserved by Hefei University of Technology.

The strains LMG 26250, NCTC 9529, ATCC 33664, cro2451A1, G362, and PAO1 in Table 1 were taken as examples to illustrate their specific sources as follows:
LMG 26250 and NCTC 9529 were standard strains purchased from Beijing BioBW Biotechnology Co., Ltd.;
ATCC 33664 was purchased from BeNa Culture Collection Co., Ltd.;

For the strain cro2451A1, reference can be made to the prior art (Zeng Haiyan, Li Chengsi, Luo Dandan, et al, Novel phage vB_CtuP_B1 for controlling Cronobacter malonaticus and Cronobacter turicensis in ready-to-eat lettuce and powered infant formula. [J]. Food Research International, 2021, 143:110255);

For the strain G362, reference can be made to the prior art (Ye YingWang, Gao Jina, Jiao Rui, et al, The Membrane Proteins Involved in Virulence of Cronobacter sakazakii Virulent G362 and Attenuated L3101 Isolates. [J]. Frontiers in microbiology, 2015, 6:01238);

For the strain PA01, reference can be made to the prior art (Klockgether Jens, Munder Antje, Neugebauer Jens, et al, Genome diversity of Pseudomonas aeruginosa PAO1 laboratory strains. [J]. Journal of bacteriology, 2010, 192(4): 1113-1121.); the sources of the remaining strains were not described in detail.

TABLE 1
Host spectrum of phage vB_CsaM_CBT2

| No. | Strain name | Species | Lysis effect |
|---|---|---|---|
| 1 | cro2375w | Cronobacter sakazakii | + |
| 2 | cro1573B3 | Cronobacter sakazakii | + |
| 3 | cro751B2 | Cronobacter sakazakii | + |
| 4 | cro3525w | Cronobacter sakazakii | + |
| 5 | cro2451A1 | Cronobacter sakazakii | + |
| 6 | cro672B3-1 | Cronobacter sakazakii | + |
| 7 | cro1931w | Cronobacter sakazakii | + |
| 8 | xzcro80 | Cronobacter sakazakii | + |
| 9 | xzcro81 | Cronobacter sakazakii | + |
| 10 | xzcro83 | Cronobacter sakazakii | + |
| 11 | xzcro90 | Cronobacter sakazakii | + |
| 12 | xzcro97 | Cronobacter sakazakii | + |
| 13 | GZcsf-1 | Cronobacter sakazakii | + |
| 14 | cro1541A1-1 | Cronobacter turicensis | + |
| 15 | cro2864C1 | Cronobacter turicensis | + |
| 16 | cro1187W | Cronobacter muytjensii | + |
| 17 | LMG 26250 | Cronobacter condimenti | + |
| 18 | NCTC 9529 | Cronobacter universalis | − |
| 19 | G362 | Cronobacter malonaticus | − |
| 20 | ATCC 33664 | Burkholderia gladioli | − |
| 21 | BG001 | Burkholderia gladioli | − |
| 22 | BG005 | Burkholderia gladioli | − |
| 23 | BG006 | Burkholderia gladioli | − |
| 24 | BG007 | Burkholderia gladioli | − |
| 25 | BG0016 | Burkholderia gladioli | − |
| 26 | BG0017 | Burkholderia gladioli | − |
| 27 | BG0018 | Burkholderia gladioli | − |
| 28 | BG0019 | Burkholderia gladioli | − |
| 29 | BG0024 | Burkholderia gladioli | − |
| 30 | WT60 | Vibrio parahemolyticus | − |
| 31 | WT64 | Vibrio parahemolyticus | − |
| 32 | WT78 | Vibrio parahemolyticus | − |
| 33 | WT80 | Vibrio parahemolyticus | − |
| 34 | WT81 | Vibrio parahemolyticus | − |
| 35 | WT83 | Vibrio parahemolyticus | − |
| 36 | WT85 | Vibrio parahemolyticus | − |
| 37 | WT89 | Vibrio parahemolyticus | − |
| 38 | WT91 | Vibrio parahemolyticus | − |
| 39 | WT92 | Vibrio parahemolyticus | − |
| 40 | WT01 | Klebsiella pneumoniae | − |
| 41 | WT02 | Klebsiella pneumoniae | − |
| 42 | WT03 | Klebsiella pneumoniae | − |
| 43 | WT04 | Klebsiella pneumoniae | − |
| 44 | WT05 | Klebsiella pneumoniae | − |
| 45 | WT06 | Klebsiella pneumoniae | − |
| 46 | WT07 | Klebsiella pneumoniae | − |
| 47 | WT08 | Klebsiella pneumoniae | − |
| 48 | WT09 | Klebsiella pneumoniae | − |
| 49 | WT10 | Klebsiella pneumoniae | − |
| 50 | 72-5 | Salmonella | − |
| 51 | 72-1 | Salmonella | − |
| 52 | E.C | Escherichia coli | − |
| 53 | 3372A1 | Escherichia coli | − |
| 54 | 3466A5 | Escherichia coli | − |
| 55 | 2627-2 | Escherichia coli | − |
| 56 | 10813 | Escherichia coli | − |
| 57 | 295 | Staphylococcus aureus | − |
| 58 | 313 | Staphylococcus aureus | − |
| 59 | 3620 | Staphylococcus aureus | − |
| 60 | PAO1 | Pseudomonas aeruginosa | − |

### Example 5 Determination of an Optimal MOI of Phage

Cronobacter GZcsf-1 cells were cultured to a pre-logarithmic phase, such that a bacterial concentration was 10⁸ cfu/mL. Different treatment groups were added with the proliferation liquid of Cronobacter phage vB_CsaM_CBT2 prepared in Example 1 and the bacterial solution of the host Cronobacter GZcsf-1 according to the MOI of 10:1, 1:1, 1:10, 1:100, 1:100, 1:1000, and 1:10000. Each group was cultured at 37° C. and 200 rpm for 4 h, centrifuged at 12,000 rpm for 10 min to 15 min, and filtered with a 0.45 μm filter head, and a titer of an obtained filtrate was measured by a double-layer agar plate method. 3 parallel experiments were set up for each treatment group. An MOI that produced the highest titer was the optimal MOI.

The results were shown in Table 2. When MOI=1:100, the highest titer was 1.18×10¹⁰ pfu/mL, that is, the optimal MOI of phage vB_CsaM_CBT2 was 1:100.

TABLE 2
Optimal MOI of phage

| MOI | Titer pfu/mL |
|---|---|
| 10:1 | 2.95 × 10⁸ |
| 1:1 | 3.17 × 10⁹ |
| 1:10 | 6.40 × 10⁹ |
| 1:100 | 1.18 × 10¹⁰ |
| 1:1000 | 1.13 × 10¹⁰ |
| 1:10000 | 7.07 × 10⁹ |

### Example 6 Determination of a One-Step Growth Curve of Phage

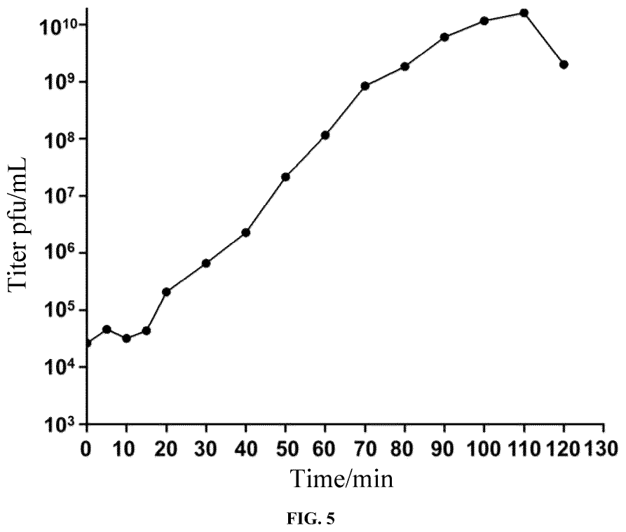

The Cronobacter phage GZcsf-1 cells were cultured to a pre-logarithmic phase, such that a bacterial concentration was 10⁸ cfu/mL, and the proliferation liquid of the Cronobacter phage vB_CsaM_CBT2 prepared in Example 1 and the host Cronobacter GZesf-1 at an optimal MOI of 1:100 were placed in a water bath at 37° C. for 5 min, centrifuged at 12,000 rpm for 30 s, and a supernatant was discarded to obtain a precipitate. The precipitate was washed twice with LB medium. 30 mL of LB medium preheated at 37° C. was added to the precipitate, and cultured at 37° C. with shaking. Samples were taken at 0 min, 5 min, 10 min, 15 min, and 20 min, and then every 10 min. Each sample was centrifuged at 12,000 rpm for 30 s, filtered through a 0.45 μm filter head, and a titer of phage at each time point was determined. 3 parallel experiments were set up. The titer was measured by a double-layer agar plate method, and the one-step growth curve was drawn with the infection time as an abscissa and the phage titer as an ordinate, as shown in Table 3 and FIG. 5.

TABLE 3
Titer of phage (pfu/mL)

| Infection time | | | | | |
|---|---|---|---|---|---|
| (min) | Parallel 1 | Parallel 2 | Parallel 3 | Average value | Deviation |
| 0 | 2.20 × 10⁴ | 2.80 × 10⁴ | 2.90 × 10⁴ | 2.63 × 10⁴ | 3.09 × 10³ |
| 5 | 4.70 × 10⁴ | 5.10 × 10⁴ | 4.00 × 10⁴ | 4.60 × 10⁴ | 4.55 × 10³ |
| 10 | 3.00 × 10⁴ | 3.10 × 10⁴ | 3.50 × 10⁴ | 3.20 × 10⁴ | 2.16 × 10³ |
| 15 | 4.10 × 10⁴ | 4.90 × 10⁴ | 4.10 × 10⁴ | 4.37 × 10⁴ | 3.77 × 10³ |
| 20 | 2.00 × 10⁵ | 2.29 × 10⁵ | 1.94 × 10⁵ | 2.08 × 10⁵ | 1.53 × 10⁴ |
| 30 | 6.50 × 10⁵ | 6.30 × 10⁵ | 7.10 × 10⁵ | 6.63 × 10⁵ | 3.40 × 10⁴ |
| 40 | 2.28 × 10⁶ | 2.29 × 10⁶ | 2.33 × 10⁶ | 2.30 × 10⁶ | 2.16 × 10⁴ |
| 50 | 2.12 × 10⁷ | 2.04 × 10⁷ | 2.32 × 10⁷ | 2.16 × 10⁷ | 1.18 × 10⁶ |
| 60 | 1.06 × 10⁸ | 1.13 × 10⁸ | 1.27 × 10⁸ | 1.15 × 10⁸ | 8.73 × 10⁶ |
| 70 | 9.10 × 10⁸ | 8.70 × 10⁸ | 7.80 × 10⁸ | 8.53 × 10⁸ | 5.44 × 10⁷ |
| 80 | 1.84 × 10⁹ | 1.83 × 10⁹ | 1.88 × 10⁹ | 1.85 × 10⁹ | 2.16 × 10⁷ |
| 90 | 6.90 × 10⁹ | 5.30 × 10⁹ | 6.10 × 10⁹ | 6.10 × 10⁹ | 6.53 × 10⁸ |
| 100 | 1.06 × 10¹⁰ | 1.19 × 10¹⁰ | 1.27 × 10¹⁰ | 1.17 × 10¹⁰ | 8.65 × 10⁸ |
| 110 | 1.61 × 10¹⁰ | 1.49 × 10¹⁰ | 1.75 × 10¹⁰ | 1.62 × 10¹⁰ | 1.06 × 10⁹ |
| 120 | 1.90 × 10⁹ | 2.11 × 10⁹ | 2.02 × 10⁹ | 2.01 × 10⁹ | 8.60 × 10⁷ |

According to FIG. 5 and Table 3, 0-20 min was the incubation period of Cronobacter phage vB_CsaM_CBT2, 20-100 min was the lysis period of Cronobacter phage vB_CsaM_CBT2, and 100-120 min was the stationary period of Cronobacter phage vB_CsaM_CBT2.

### Example 7 Determination of a Temperature Stability of Cronobacter Phage vB_CsaM_CBT2

1 mL of the proliferation liquid of the Cronobacter phage vB_CsaM_CBT2 with a titer of 10¹⁰ pfu/mL was placed in a 1.5 mL centrifuge tube and incubated in a water bath at 25° C. for 1 h. After the incubation, the phage was taken out and cooled on ice. The titer was measured by a double-layer agar plate method. 3 parallel experiments were set up.

### Example 8

1 mL of the proliferation liquid of the Cronobacter phage vB_CsaM_CBT2 with a titer of 10¹⁰ pfu/mL was placed in a 1.5 mL centrifuge tube and incubated in a water bath at 30° C. for 1 h. After the incubation, the phage was taken out and cooled on ice. The titer was measured by a double-layer agar plate method. 3 parallel experiments were set up.

### Example 9

1 mL of the proliferation liquid of the Cronobacter phage vB_CsaM_CBT2 with a titer of 10¹⁰ pfu/mL was placed in a 1.5 mL centrifuge tube and incubated in a water bath at 37° C. for 1 h. After the incubation, the phage was taken out and cooled on ice. The titer was measured by a double-layer agar plate method. 3 parallel experiments were set up.

### Example 10

1 mL of the proliferation liquid of the Cronobacter phage vB_CsaM_CBT2 with a titer of 10¹⁰ pfu/mL was placed in a 1.5 mL centrifuge tube and incubated in a water bath at 40° C. for 1 h. After the incubation, the phage was taken out and cooled on ice. The titer was measured by a double-layer agar plate method. 3 parallel experiments were set up.

### Example 11

1 mL of the proliferation liquid of the Cronobacter phage vB_CsaM_CBT2 with a titer of 10¹⁰ pfu/mL was placed in a 1.5 mL centrifuge tube and incubated in a water bath at 50° C. for 1 h. After the incubation, the phage was taken out and cooled on ice. The titer was measured by a double-layer agar plate method. 3 parallel experiments were set up.

### Example 12

1 mL of the proliferation liquid of the Cronobacter phage vB_CsaM_CBT2 with a titer of 10¹⁰ pfu/mL was placed in a 1.5 mL centrifuge tube and incubated in a water bath at 60° C. for 1 h. After the incubation, the phage was taken out and cooled on ice. The titer was measured by a double-layer agar plate method. 3 parallel experiments were set up.

### Example 13

1 mL of the proliferation liquid of the Cronobacter phage vB_CsaM_CBT2 with a titer of 10¹⁰ pfu/mL was placed in a 1.5 mL centrifuge tube and incubated in a water bath at 65° C. for 1 h. After the incubation, the phage was taken out and cooled on ice. The titer was measured by a double-layer agar plate method. 3 parallel experiments were set up.

### Example 14

1 mL of the proliferation liquid of the Cronobacter phage vB_CsaM_CBT2 with a titer of 10¹⁰ pfu/mL was placed in a 1.5 mL centrifuge tube and incubated in a water bath at 70° C. for 1 h. After the incubation, the phage was taken out and cooled on ice. The titer was measured by a double-layer agar plate method. 3 parallel experiments were set up.

### Comparative Example 1

1 mL of the proliferation liquid of the Cronobacter phage vB_CsaM_CBT2 with a titer of 10¹⁰ pfu/mL was placed in a 1.5 mL centrifuge tube and incubated in a water bath at 75° C. for 1 h. After the incubation, the phage was taken out and cooled on ice. The titer was measured by a double-layer agar plate method. 3 parallel experiments were set up.

### Comparative Example 2

1 mL of the proliferation liquid of the Cronobacter phage vB_CsaM_CBT2 with a titer of 10¹⁰ pfu/mL was placed in a 1.5 mL centrifuge tube and incubated in a water bath at 80° C. for 1 h. After the incubation, the phage was taken out and cooled on ice. The titer was measured by a double-layer agar plate method. 3 parallel experiments were set up.

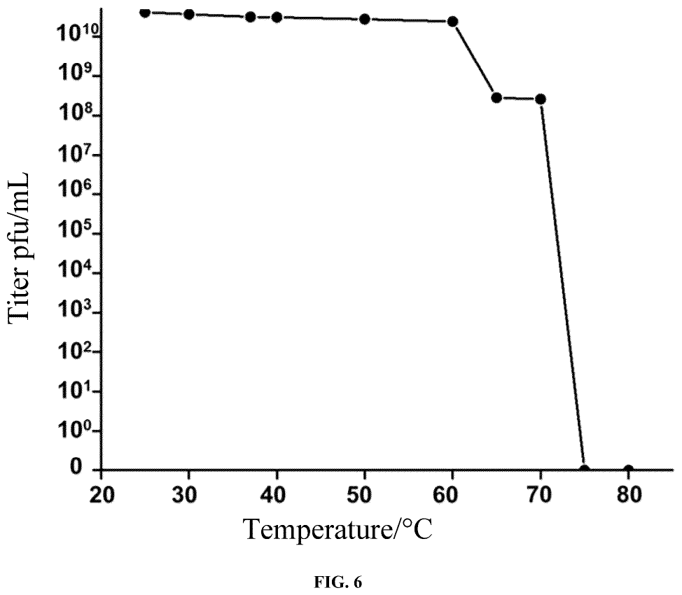

The titer determination results of the phages incubated for 1 h at different temperatures in Examples 7 to 14 and Comparative Examples 1 to 2 are shown in Table 4 and FIG. 6.

TABLE 4
Titer determination results of phages incubated
at different temperatures for 1 h (pfu/mL)

| Temperature | | | | | |
|---|---|---|---|---|---|
| (° C.) | Parallel 1 | Parallel 2 | Parallel 3 | Average value | Deviation |
| 25 | 4.36 × 10¹⁰ | 4.25 × 10¹⁰ | 3.97 × 10¹⁰ | 4.19 × 10¹⁰ | 1.64 × 10⁹ |
| 30 | 3.63 × 10¹⁰ | 4.06 × 10¹⁰ | 3.55 × 10¹⁰ | 3.75 × 10¹⁰ | 2.24 × 10⁹ |
| 37 | 3.32 × 10¹⁰ | 3.28 × 10¹⁰ | 3.02 × 10¹⁰ | 3.21 × 10¹⁰ | 1.33 × 10⁹ |
| 40 | 3.14 × 10¹⁰ | 3.28 × 10¹⁰ | 3.01 × 10¹⁰ | 3.14 × 10¹⁰ | 1.10 × 10⁹ |
| 50 | 2.81 × 10¹⁰ | 2.94 × 10¹⁰ | 2.74 × 10¹⁰ | 2.83 × 10¹⁰ | 8.29 × 10⁸ |
| 60 | 2.63 × 10¹⁰ | 2.31 × 10¹⁰ | 2.42 × 10¹⁰ | 2.45 × 10¹⁰ | 1.33 × 10⁹ |
| 65 | 2.82 × 10⁸ | 2.74 × 10⁸ | 2.92 × 10⁸ | 2.83 × 10⁸ | 7.36 × 10⁶ |
| 70 | 2.72 × 10⁸ | 2.60 × 10⁸ | 2.48 × 10⁸ | 2.60 × 10⁸ | 9.80 × 10⁶ |
| 75 | 0 | 0 | 0 | 0 | 0 |
| 80 | 0 | 0 | 0 | 0 | 0 |

According to Table 4 and FIG. 6, the titer of the Cronobacter phage vB_CsaM_CBT2 hardly changed after being exposed to 25° C. to 70° C. for 1 h. After the phage vB_CsaM_CBT2 was exposed to 65° C. and 70° C. for 1 h, the titer decreased by about 8.4 log pfu/mL. After acting at 75° C. and 80° C. for 1 h, the phage vB_CsaM_CBT2 was completely inactivated.

### Example 15

100 μL of the proliferation liquid of the Cronobacter phage vB_CsaM_CBT2 with a titer of 10¹⁰ pfu/mL was added into a 1.5 mL centrifuge tube, added with 900 μL of LB broth medium with a pH value of 3, the centrifuge tube was placed in a 37° C. water bath for 1 h of culture, and then the phase was taken out. The titer was measured by a double-layer agar plate method. 3 parallel experiments were set up.

### Example 16

This example is basically the same as Example 15 but differs only in that 900 μL of LB broth medium with a pH value of 4 is added.

### Example 17

This example is basically the same as Example 15 but differs only in that 900 μL of LB broth medium with a pH value of 5 is added.

### Example 18

This example is basically the same as Example 15 but differs only in that 900 μL of LB broth medium with a pH value of 6 is added.

### Example 19

This example is basically the same as Example 15 and differs only in that 900 μL of LB broth medium with a pH value of 7 is added.

### Example 20

This example is basically the same as Example 15 and differs only in that 900 μL of LB broth medium with a pH value of 8 is added.

### Example 21

This example is basically the same as Example 15 and differs only in that 900 μL of LB broth medium with a pH value of 9 is added.

### Example 22

This example is basically the same as Example 15 and differs only in that 900 μL of LB broth medium with a pH value of 10 is added.

### Example 23

This example is basically the same as Example 15 and differs only in that 900 μL of LB broth medium with a pH value of 11 is added.

### Comparative Example 3

This example is basically the same as Example 15 and differs only in that 900 μL of LB broth medium with a pH value of 2 is added.

### Comparative Example 4

This example is basically the same as Example 15 and differs only in that 900 μL of LB broth medium with a pH value of 12 is added.

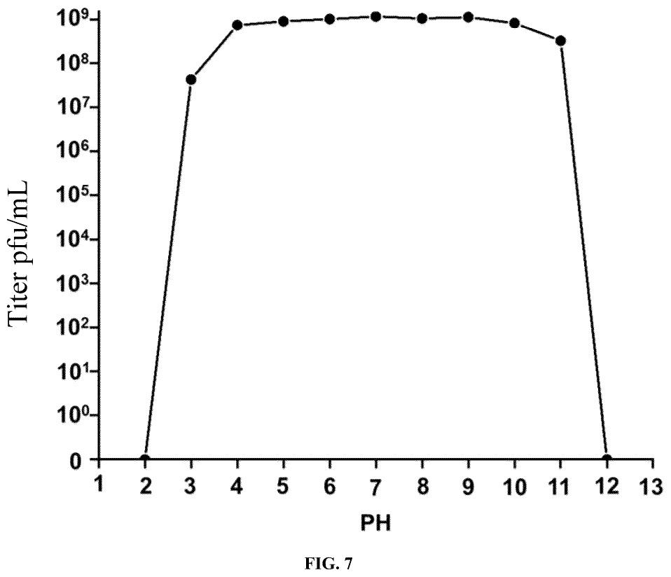

The titer determination results of the phages incubated for 1 h at different temperatures in Examples 15 to 23 and Comparative Examples 3 to 4 are shown in Table 5 and FIG. 7.

TABLE 5
Titer determination results of phages incubated
at different pH values for 1 h (pfu/mL)

| pH | Parallel 1 | Parallel 2 | Parallel 3 | Average value | Deviation |
|---|---|---|---|---|---|
| 2 | 0 | 0 | 0 | 0 | 0 |
| 3 | 4.80 × 10⁷ | 4.00 × 10⁷ | 4.10 × 10⁷ | 4.30 × 10⁷ | 3.56 × 10⁶ |
| 4 | 7.10 × 10⁸ | 8.30 × 10⁸ | 6.90 × 10⁸ | 7.43 × 10⁸ | 6.18 × 10⁷ |
| 5 | 8.70 × 10⁸ | 8.50 × 10⁸ | 1.00 × 10⁹ | 9.07 × 10⁸ | 6.65 × 10⁷ |
| 6 | 8.30 × 10⁸ | 1.10 × 10⁹ | 1.13 × 10⁹ | 1.02 × 10⁹ | 1.35 × 10⁸ |
| 7 | 1.08 × 10⁹ | 1.40 × 10⁹ | 1.01 × 10⁹ | 1.16 × 10⁹ | 1.70 × 10⁸ |
| 8 | 1.33 × 10⁹ | 9.20 × 10⁸ | 9.10 × 10⁸ | 1.05 × 10⁹ | 1.96 × 10⁸ |
| 9 | 1.19 × 10⁹ | 1.21 × 10⁹ | 9.80 × 10⁸ | 1.12 × 10⁹ | 1.04 × 10⁸ |
| 10 | 8.10 × 10⁸ | 7.90 × 10⁸ | 8.80 × 10⁸ | 8.27 × 10⁸ | 3.86 × 10⁷ |
| 11 | 2.90 × 10⁸ | 3.70 × 10⁸ | 3.20 × 10⁸ | 3.27 × 10⁸ | 3.30 × 10⁷ |
| 12 | 0 | 0 | 0 | 0 | 0 |

As shown in Table 5 and FIG. 7, the titer of Cronobacter phage vB_CsaM_CBT2 hardly changed at a pH value of 3 to 11. At pH values of 2 and 12, the phage vB_CsaM_CBT2 was completely inactivated.

### Example 24 Bactericidal Effect of Phage in Reconstituted Milk Powder

Three brands of PIFs were selected for testing, and each milk powder was an aseptic milk powder. The milk powder was dissolved in sterile water to obtain a milk powder sample with a mass concentration of 10%. The bacterial solution of Cronobacter GZcsf-1 was added to the milk powder sample to construct an experimental treatment model. For Cronobacter GZcsf-1, reference can be made to the prior art (Zeng Haiyan, Lei Tao, He Wenjing, et al. Novel Multidrug-Resistant Cronobacter sakazakii Causing Meningitis in Neonate, China, 2015. [J]. Emerging infectious diseases, 2018, 24(11): 2121.).

Experimental group: 500 μL of bacterial solution of 105 cfu/mL Cronobacter GZcsf-1 and 500 μL of proliferation liquid of 10¹⁰ pfu/mL phage prepared in Example 1 were added in 25 mL of milk powder sample, and mixed well. A resulting mixture was cultured at 4° C. and 37° C. at 200 rpm, and samples were taken at 0 h, 2 h, 4 h, 6 h, and 12 h, and a bactericidal effect was detected by determining the number of bacteria through a plate counting method. 3 parallel experiments were set up for each treatment when being cultured at 4° C. and 37° C.

Control group: sterile physiological saline was used instead of the phage proliferation liquid as a control group, and 3 parallel experiments were set up for the control group.

The bactericidal rate of phage vB_CsaM_CBT2 to Cronobacter was calculated according to the following formula:

The bactericidal rate of phage vB_CsaM_CBT2 to Cronobacter (%)=(amount of host bacteria in control group−amount of host bacteria in experimental group)/amount of host bacteria in control group.

The results are shown in Table 6 and Table 7. After culturing at 4° C. for 6 h, the bactericidal rate of Cronobacter phage vB_CsaM_CBT2 on brand 1 milk powder samples was about 98.60%. The bactericidal rate of Cronobacter in brand 2 milk powder samples was about 77.86%. The bactericidal rate of Cronobacter in brand 3 milk powder samples was about 71.87%; the bactericidal rate of Cronobacter phage vB_CsaM_CBT2 in brand 1 milk powder samples was about 99.96% when cultured at 37° C. and 200 rpm for 2 h. The bactericidal rate of Cronobacter in brand 2 milk powder samples was about 99.84%. The bactericidal rate of Cronobacter in brand 3 milk powder samples was about 99.97%. Overall, the Cronobacter phage vB_CsaM_CBT2 had a better killing effect on Cronobacter at 37° C. than at 4° C., especially when cultured at 37° C. for 2 h, the Cronobacter phage vB_CsaM_CBT2 could kill more than 99.9% of Cronobacter in milk powder samples. This showed that the Cronobacter phage vB_CsaM_CBT2 could effectively kill Cronobacter in PIF.

TABLE 6
Number of Cronobacter in different treatment groups
of different brands of milk powder (unit: CFU/mL)

| | Treat | | Treatment | | | Number of Cronobacter | | |
|---|---|---|---|---|---|---|---|---|
| PIF | temperature | | time | | Group | Parallel 1 | Parallel 2 | Parallel 3 |
| Brand 1 | 4° | C. | 2 | h | Control | 1.77 × 10⁴ | 1.70 × 10⁴ | 1.79 × 10⁴ |
| | | | | | Experimental | 5.60 × 10³ | 7.20 × 10³ | 5.70 × 10³ |
| | | | | | group | | | |
| | | | 4 | h | Control | 1.59 × 10⁴ | 1.64 × 10⁴ | 1.87 × 10⁴ |
| | | | | | Experimental | 6.70 × 10³ | 5.90 × 10³ | 5.70 × 10³ |
| | | | | | group | | | |
| | | | 6 | h | Control | 2.21 × 10⁴ | 2.10 × 10⁴ | 2.34 × 10⁴ |
| | | | | | Experimental | 365 | 249 | 317 |
| | | | | | group | | | |
| | | | 12 | h | Control | 4.35 × 10⁴ | 4.01 × 10⁴ | 3.82 × 10⁴ |
| | | | | | Experimental | 258 | 283 | 248 |
| | | | | | group | | | |
| | 37° | C. | 2 | h | Control | 1.07 × 10⁵ | 1.32 × 10⁵ | 1.15 × 10⁵ |
| | | | | | Experimental | 46 | 45 | 48 |
| | | | | | group | | | |
| | | | 4 | h | Control | 1.37 × 10⁷ | 1.52 × 10⁷ | 1.61 × 10⁷ |
| | | | | | Experimental | 2.77 × 10⁴ | 3.00 × 10⁴ | 2.81 × 10⁴ |
| | | | | | group | | | |
| | | | 6 | h | Control | 1.54 × 10⁸ | 1.61 × 10⁸ | 1.5 × 10⁸ |
| | | | | | Experimental | 2.55 × 10⁵ | 2.73 × 10⁵ | 2.82 × 10⁵ |
| | | | | | group | | | |
| | | | 12 | h | Control | 6.40 × 10⁸ | 6.30 × 10⁸ | 7.70 × 10⁸ |
| | | | | | Experimental | 5.20 × 10⁷ | 4.80 × 10⁷ | 6.00 × 10⁷ |
| | | | | | group | | | |
| Brand 2 | 4° | C. | 2 | h | Control | 2.41 × 10⁴ | 2.39 × 10⁴ | 2.47 × 10⁴ |
| | | | | | Experimental | 1.30 × 10⁴ | 1.36 × 10⁴ | 1.44 × 10⁴ |
| | | | | | group | | | |
| | | | 4 | h | Control | 2.31 × 10⁴ | 2.44 × 10⁴ | 2.88 × 10⁴ |
| | | | | | Experimental | 1.66 × 10⁴ | 2.08 × 10⁴ | 2.02 × 10⁴ |
| | | | | | group | | | |
| | | | 6 | h | Control | 8.00 × 10⁴ | 7.50 × 10⁴ | 7.40 × 10⁴ |
| | | | | | Experimental | 1.79 × 10⁴ | 1.60 × 10⁴ | 1.68 × 10⁴ |
| | | | | | group | | | |
| | | | 12 | h | Control | 1.16 × 10⁵ | 8.60 × 10⁴ | 9.70 × 10⁴ |
| | | | | | Experimental | 8.50 × 10⁴ | 7.90 × 10⁴ | 6.80 × 10⁴ |
| | | | | | group | | | |
| | 37° | C. | 2 | h | Control | 1.12 × 10⁵ | 8.20 × 10⁴ | 1.01 × 10⁵ |
| | | | | | Experimental | 160 | 149 | 162 |
| | | | | | group | | | |
| | | | 4 | h | Control | 1.38 × 10⁷ | 1.43 × 10⁷ | 1.39 × 10⁷ |
| | | | | | Experimental | 1.64 × 10⁴ | 1.66 × 10⁴ | 1.67 × 10⁴ |
| | | | | | group | | | |
| | | | 6 | h | Control | 2.69 × 10⁸ | 2.46 × 10⁸ | 2.59 × 10⁸ |
| | | | | | Experimental | 3.84 × 10⁶ | 3.52 × 10⁶ | 2.56 × 10⁶ |
| | | | | | group | | | |
| | | | 12 | h | Control | 1.19 × 10⁹ | 1.16 × 10⁹ | 1.17 × 10⁹ |
| | | | | | Experimental | 2.25 × 10⁸ | 1.72 × 10⁸ | 1.75 × 10⁸ |
| | | | | | group | | | |
| Brand 3 | 4° | C. | 2 | h | Control | 2.61 × 10⁴ | 2.55 × 10⁴ | 2.64 × 10⁴ |
| | | | | | Experimental | 1.65 × 10⁴ | 1.43 × 10⁴ | 1.75 × 10⁴ |
| | | | | | group | | | |
| | | | 4 | h | Control | 3.00 × 10⁴ | 2.84 × 10⁴ | 2.86 × 10⁴ |
| | | | | | Experimental | 1.72 × 10⁴ | 1.60 × 10⁴ | 1.42 × 10⁴ |
| | | | | | group | | | |
| | | | 6 | h | Control | 7.60 × 10⁴ | 6.80 × 10⁴ | 8.00 × 10⁴ |
| | | | | | Experimental | 2.60 × 10⁴ | 3.30 × 10⁴ | 2.80 × 10⁴ |
| | | | | | group | | | |
| | | | 12 | h | Control | 1.34 × 10⁵ | 1.57 × 10⁵ | 1.47 × 10⁵ |
| | | | | | Experimental | 4.50 × 10⁴ | 3.60 × 10⁴ | 4.10 × 10⁴ |
| | | | | | group | | | |
| | 37° | C. | 2 | h | Control | 1.19 × 10⁵ | 1.86 × 10⁵ | 1.67 × 10⁵ |
| | | | | | Experimental | 36 | 48 | 52 |
| | | | | | group | | | |
| | | | 4 | h | Control | 1.33 × 10⁷ | 1.49 × 10⁷ | 1.36 × 10⁷ |
| | | | | | Experimental | 2.40 × 10⁴ | 2.10 × 10⁴ | 3.20 × 10⁴ |
| | | | | | group | | | |
| | | | 6 | h | Control | 3.90 × 10⁷ | 2.80 × 10⁷ | 2.00 × 10⁷ |
| | | | | | Experimental | 2.91 × 10⁵ | 2.59 × 10⁵ | 3.47 × 10⁵ |
| | | | | | group | | | |
| | | | 12 | h | Control | 3.20 × 10⁸ | 4.90 × 10⁸ | 3.40 × 10⁸ |
| | | | | | Experimental | 6.00 × 10⁷ | 1.22 × 10⁸ | 5.00 × 10⁷ |
| | | | | | group | | | |

TABLE 7
Bactericidal rate of phage vB_CsaM_CBT2 on Cronobacter

| PIF | Time (h) | 4° C. (%) | 37° C. (%) |
|---|---|---|---|
| Brand 1 | 2 | 64.72 ± 5.00 | 99.96 ± 0.00 |
| | 4 | 63.80 ± 4.76 | 99.81 ± 0.01 |
| | 6 | 98.60 ± 0.19 | 99.83 ± 0.01 |
| | 12 | 99.35 ± 0.05 | 92.15 ± 0.21 |
| Brand 2 | 2 | 43.62 ± 1.82 | 99.84 ± 0.02 |
| | 4 | 24.25 ± 6.75 | 99.88 ± 0.00 |
| | 6 | 77.86 ± 0.58 | 98.72 ± 0.21 |
| | 12 | 21.59 ± 9.60 | 83.77 ± 1.89 |
| Brand 3 | 2 | 38.14 ± 4.28 | 99.97 ± 0.00 |
| | 4 | 45.56 ± 3.41 | 99.81 ± 0.04 |
| | 6 | 60.75 ± 6.57 | 98.86 ± 0.43 |
| | 12 | 71.87 ± 4.35 | 80.55 ± 4.19 |

In summary, the Cronobacter phage vB_CsaM_CBT2 provided by the present disclosure had desirable stability and high bactericidal effect at a pH value of 3 to 11 and 25° C. to 70° C. Moreover, the phage did not carry any virulence and antibiotic resistance genes, and met the safety requirements in practical applications. The phage had a bactericidal effect of 80.55% to 99.97% in milk powder samples within 12 h. The phage provides a new strategy and resource guarantee for the control of Cronobacter contamination in PIF and its industrial chain and environment.

Although the above example has described the present disclosure in detail, it is only a part of, not all of, the examples of the present disclosure. Other examples may also be obtained by persons based on the example without creative efforts, and all of these examples shall fall within the protection scope of the present disclosure.

1. A Cronobacter phage vB_CsaM_CBT2, wherein the phage has a deposit number of CCTCC NO: M 2023524.

2. A method for inhibiting and/or killing Cronobacter, comprising a step of contacting the Cronobacter phage vB_CsaM_CBT2 according to claim 1 with Cronobacter.

3. The method according to claim 2, wherein the preparation is prepared in the form of a proliferation liquid of the Cronobacter phage vB_CsaM_CBT2 or a concentrate of the proliferation liquid.

4. The method according to claim 3, wherein a preparation method of the proliferation liquid comprises the following steps:
mixing a bacterial solution in a logarithmic phase of the Cronobacter and the Cronobacter phage vB_CsaM_CBT2 with an LB broth medium, conducting cell culture to obtain a culture solution; subjecting the culture solution to centrifugation to obtain a supernatant; and subjecting the supernatant to filtration to obtain the proliferation liquid of the Cronobacter phage vB_CsaM_CBT2.

5. The method according to claim 2, wherein the Cronobacter is one or more selected from the group consisting of Cronobacter sakazakii, Cronobacter turicensis, Cronobacter muytjensii, and Cronobacter condimenti.

6. The method according to claim 2, wherein the Cronobacter phage vB_CsaM_CBT2 in the preparation for inhibiting the Cronobacter has a working titer of greater than or equal to 2.95×10⁸ pfu/mL; and the Cronobacter phage vB_CsaM_CBT2 in the preparation for killing the Cronobacter has a working titer of greater than or equal to 1×10¹⁰ pfu/mL.

7. An inhibitor or a bactericide of Cronobacter, comprising the Cronobacter phage vB_CsaM_CBT2 according to claim 1 and a pharmaceutically acceptable excipient.

8. The inhibitor or the bactericide of the Cronobacter according to claim 7, wherein the inhibitor or the bactericide of the Cronobacter has a working pH value of 3 to 11.

9. The inhibitor or the bactericide of the Cronobacter according to claim 7, wherein the inhibitor of the Cronobacter has a working titer of greater than or equal to 2.95×10⁸ pfu/mL; and the bactericide of the Cronobacter has a working titer of greater than or equal to 1×10¹⁰ pfu/mL.

10. The inhibitor or the bactericide of the Cronobacter according to claim 7, wherein the pharmaceutically acceptable excipient is one or more selected from the group consisting of a dispersant, a stabilizer, a filler, and a solvent.

11. A method for controlling Cronobacter contamination, comprising a step of applying the inhibitor or the bactericide of the Cronobacter according to claim 7.

12. The method according to claim 11, wherein the reagent has a multiplicity of infection (MOI) of 1:10 to 1:1000.

13. The method according to claim 11, wherein a process of controlling the Cronobacter contamination comprises antagonizing the Cronobacter contamination in a product and/or an environment.

14. The method according to claim 13, wherein the product comprises a food product.

15. The method according to claim 3, wherein the Cronobacter phage vB_CsaM_CBT2 in the preparation for inhibiting the Cronobacter has a working titer of greater than or equal to 2.95×10⁸ pfu/mL; and the Cronobacter phage vB_CsaM_CBT2 in the preparation for killing the Cronobacter has a working titer of greater than or equal to 1×10¹⁰ pfu/mL.

16. The method according to claim 11, wherein inhibitor or the bactericide of the Cronobacter has a working pH value of 3 to 11.

17. The method according to claim 11, wherein the inhibitor of the Cronobacter has a working titer of greater than or equal to 2.95×10⁸ pfu/mL; and the bactericide of the Cronobacter has a working titer of greater than or equal to 1×10¹⁰ pfu/mL.

18. The method according to claim 11, wherein the pharmaceutically acceptable excipient is one or more selected from the group consisting of a dispersant, a stabilizer, a filler, and a solvent.
